(19) United States
(12) Patent Application Publication
(10) Pub. No.: US 2026/0226514 A1
(43) Pub. Date: Aug. 6, 2026

(54) FAT/OIL PRODUCTION METHOD USING YEAST BELONGING TO GENUS METSCHNIKOWIA AND NOVEL METSCHNIKOWIA YEAST USED THEREIN

(71) Applicant: IDEMITSU KOSAN CO.,LTD., Tokyo (JP)

(72) Inventors: Shinzo MAYUZUMI, Tokyo (JP); Tomoko ISHII, Tokyo (JP); Shuichiro KIMURA, Tokyo (JP)

(73) Assignee: IDEMITSU KOSAN CO.,LTD., Tokyo (JP)

(21) Appl. No.: 19/142,503

(22) PCT Filed: Dec. 22, 2023
(86) PCT No.: PCT/JP2023/046269

§ 371 Date: Jun. 23, 2025

(30) Foreign Application Priority Data
Dec. 23, 2022 (JP) 2022-207031

(51) Int. Cl. C12P 7/64; C12N 1/165; C12N 1/18; C12R 1/645

(52) U.S. Cl. CPC C12P 7/64; C12N 1/165; C12N 1/18; C12P 2203/00; C12R 2001/645

(57) ABSTRACT

A Metschnikowia sp. IS-391 strain (NITE BP-03676) or a related strain is cultured in a medium containing sugar to produce fats and oils from sugar, followed by collection of the resulting fats and oils.

### TECHNICAL FIELD

The present invention relates to a method of producing fats and oils using yeast and a novel yeast used in the method.

### BACKGROUND TECHNOLOGY

Vegetable oils and animal fats have been widely used as green fuel materials so far, but competition with food and the destruction of tropical rainforests due to palm plantations are becoming increasingly problematic.

Therefore, the production of fats and oils from lignocellulosic biomass such as agricultural residues by microalgae or heterotrophic microorganisms (yeast and molds) has been attracting attention. However, the high cost of both of these methods is an issue, and in order to overcome this issue, it is necessary to improve the production efficiency (oil-to-sugar yield) of fats and oils produced by heterotrophic microorganisms. In addition, resistance to growth inhibitors generated during saccharification of lignocellulose is necessary.

Non-patent literature 1, 2 and 3 disclose that yeasts of the genus Metschnikowia are capable of producing oil and fat. Non-Patent Document 2 reports that Metschnikowia pulcherrima is resistant to growth inhibitors in lignocellulose saccharified solution, but Metschnikowia yeast is inferior to oleaginous yeasts of other genera such as Lipomyces in terms of oleaginous productivity.

### PRIOR ART REFERENCES

### Non-Patent Documents

Non-Patent Document 1: Santamauro et al. (2014) Biotechnol Biofuels. 2014; 7: 34. doi: 10.1186/1754-6834-7-34
Non-patent document 2: Fraeya (2016) A palm oil substitute and care product emulsions from a yeast cultivated on waste resources, PhD Thesis, University of Bath, ISNI: 0000 0004 5918 4115
Non-Patent Document 3: Miranda et al. (2020) BMC Microbiology 20:60, DOI 10.1186/s12866-020-01742-6

### SUMMARY OF INVENTION

### Problem to be Solved by the Invention

As mentioned above, some strains of Metschnikowia yeast have been reported to be capable of producing fats and oils and to be resistant to growth inhibitors in lignocellulose-saccharified solution, but they have a relatively low oil-to-sugar yield compared to other oleaginous yeasts.

Therefore, the present invention is to provide a strain of yeast in the genus Metschnikowia with high fats and oils productivity, and to provide a method of producing fats and oils using such yeast.

### Means to Solve the Problem

As a result of the intensive study to solve the above problem, we found a strain (IS-391 strain) in Metschnikowia that has the equivalent growth inhibitor resistance as M. pulcherrima and a high oil production capacity. We also found that the strain and its related strains can be cultured in a sugar-containing medium to efficiently produce oils and fats. Based on these findings, the present invention was completed.

The present invention relates to a method for producing fats and oils, comprising culturing Metschnikowia sp. IS-391 strain (NITE BP-03676) or a related strain thereof in a medium containing sugar to produce fats and oils from sugar and collecting the resulting fats and oils.

Here, the medium containing the sugar may be a medium containing saccharified product of lignocellulosic biomass.

The culture temperature may be 10° C. to 30° C.

The molar ratio of the initial carbon source concentration to the initial nitrogen source concentration (C/N ratio) in the medium may be 100 or greater.

The related strain may be a strain whose sequence of the 26S rDNA D1/D2 region is at least 95% identical to SEQ ID NO: 1 and has oil and fat production ability equivalent to that of IS-391 strain.

The invention also provides the Metschnikowia sp. IS-391 strain (NITE BP-03676).

The fats and oils obtained by the production method can be used as raw materials for biofuels, bionaphtha, food oil, lubricating oil, or surfactants.

The invention also relates to fats and oils produced by the aforementioned production method and biofuels or bionaphtha made from said fats and oils.

### Advantageous Effects of the Invention

By using Metschnikowia sp. IS-391 strain or its related strains, oils and fats can be efficiently produced from biomass saccharified liquid and other sugars. The oils and fats produced by this method can be used not only as biofuel raw materials and bionaphtha raw materials with greenhouse gas reduction effects, but also as raw materials for food oils, lubricant base materials, and surfactants with low environmental impact.

### BRIEF DESCRIPTION OF THE DRAWINGS

FIG. 1 Microscopic image of IS-391 strain (photograph).

FIG. 2 Figure showing the phylogenetic position of the novel oleaginous yeast IS-391 strain in the molecular phylogenetic tree of the genus Metschnikowia (26S rDNAD1/D2 region sequence, NJ method). The strain culture collection number and GenBank Accession number of the DNA sequence used in the phylogenetic analysis are shown in the figure; the DNA sequence of the NBRC strain was taken from the NBTC Online Catalogue (https://www.nite.go.jp/nbrc/catalogue/). The numbers in each node are bootstrap values of 1,000 replications. The strains used in Example 1 are shown in bold.

FIG. 3 Figure showing the triacylglyceride production of each strain of Metschnikowia genus after 60 hours of culture at 28° C.

FIG. 4 Figure showing CYM medium-based nitrogen source evaluation, showing changes over time in dry cell weight in flask culture.

FIG. 5 Figure showing CYM medium-based nitrogen source evaluation, showing changes over time in triacylglyceride production in flask culture.

FIG. 6 Figure showing the evaluation of ammonium sulfate concentration based on CYM medium and the change over time of dry cell weight in flask culture.

FIG. 7 Figure showing the evaluation of ammonium sulfate concentration based on CYM medium and the changes over time of triacylglyceride production in flask culture.

FIG. 8 Figure showing the evaluation of urea concentration based on CYM medium and the changes over time in dry cell weight in flask culture.

FIG. 9 Figure showing the evaluation of urea concentration based on CYM medium and changes over time in triacylglyceride production in flask culture.

FIG. 10 Figure showing the evaluation of culture temperature and changes over time in dry cell weight in flask culture.

FIG. 11 Figure showing the evaluation of culture temperature and the change in glucose concentration over time in flask culture.

FIG. 12 Figure showing the evaluation of culture temperature and changes over time in triacylglyceride production in flask culture.

FIG. 13 Figure showing the evaluation of culture pH conditions, and changes over time in dry cell weight in 250 mL jar fermenter culture.

FIG. 14 Figure showing the evaluation of culture pH conditions and changes over time in triacylglyceride production in 250 mL jar fermenter culture.

FIG. 15 Figure showing the evaluation of culture pH conditions and changes over time in dry cell weight, glucose concentration, and triacylglyceride production in a 250 mL jar fermenter culture.

FIG. 16 Figure showing changes over time in dry cell weight and triacylglyceride production in fed-batch culture in 2 L jar fermenters.

FIG. 17 Figure showing changes over time in glucose and ethanol concentrations in fed-batch culture in 2 L jar fermenters.

FIG. 18 Figure showing changes over time in dry cell weight in a 250 mL jar fermenter in a batch culture under each initial glucose concentration condition (Example 10).

FIG. 19 Figure showing changes over time of glucose concentration in each initial glucose concentration condition in a batch culture in a 250 mL jar fermenter (Example 10).

FIG. 20 Figure showing changes over time in triacylglyceride production for each initial glucose concentration condition in a batch culture in a 250 mL jar fermenter (Example 10).

FIG. 21 Figure showing changes over time of dry cell weight in a 250 mL jar fermenter in a batch culture under each initial glucose concentration condition (Example 11).

FIG. 22 Figure showing changes in glucose concentration over time for each initial glucose concentration condition in a batch culture in a 250 mL jar fermenter (Example 11).

FIG. 23 Figure showing changes in ethanol concentration over time for each initial glucose concentration condition in a batch culture in a 250 mL jar fermenter (Example 11).

FIG. 24 Figure showing changes over time in triacylglyceride production for each initial glucose concentration condition in a batch culture in a 250 mL jar fermenter (Example 11).

### EMBODIMENTS FOR CARRYING OUT THE INVENTION

The method for producing fats and oils comprises culturing Metschnikowia sp. IS-391 strain (hereinafter simply referred to as IS-391 strain) or a related strain thereof in a medium containing sugar to produce fats and oils from the sugar, and collecting the resulting fats and oils.

Fats and oils, also called acylglycerols, produced by fermentation from sugars by Metschnikowia sp. IS-391 strain or its related strains, are not restricted to any type of fat or oil, and includes triacylglycerol, diacylglycerol, and monoacylglycerol.

Metschnikowia sp. IS-391 strain was isolated from the flowers of Trifolium pretense in Sodegaura City, Chiba Prefecture, Japan, and was deposited at NITE Patent Microorganisms Depository, National Institute of Technology and Evaluation (NITE) (Room 122, 2-5-8, Kazusa-Kamashi, Kisarazu-shi, Chiba, Japan) on Jun. 24, 2022, under the deposition number NITE BP-03676 in accordance with the Budapest Treaty.

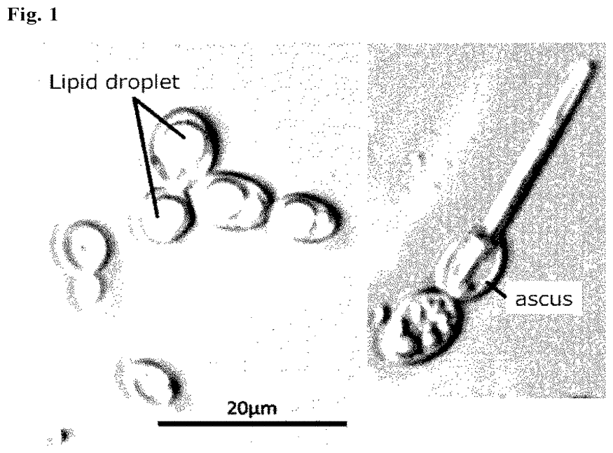

Colonies are white to white-yellow, shiny, and smooth on YPD agar medium. Cells are ovoid, frequently forming thick-film sporangia with oil droplets. Like other Metschnikowia genera, they form markedly protuberant cysts and form cystospores on the stigma (FIG. 1).

The nucleotide sequence of the D1/D2 region of the 26S rDNA region of the DNA encoding the rRNA of IS-391 strain is shown in SEQ ID NO: 1.

A related strain of IS-391 strain means a strain in which the nucleotide sequence of the D1/D2 region of the 26S rDNA region is at least 98%, preferably 99%, identical to SEQ ID NO: 1.

The related strain of IS-391 strain that can be used in the method for producing fats and oils of the present invention is a strain that has the equivalent fats and oils production ability as IS-391 strain. The term “having fats and oils production ability equivalent to IS-391 strain” means that the amount of fats and oils produced when a related strain of IS-391 strain is cultured at 28° C. in a sugar-containing medium is 80% or more, preferably 90% or more, of the amount of fats and oils produced when IS-391 strain is cultured at 28° C. in the same sugar-containing medium for the same period.

The medium is not limited as long as it contains sugar and is capable of growing Metschnikowia sp. IS-391 or related strains.

The sugar may be monosaccharides or polysaccharides. The medium may also contain sugar-containing raw materials. Various sugars can be used, including glucose, sucrose, fructose, xylose, mannose, soluble starch, glycerol, mannitol, and others, mannitol, etc.

Examples of sugar-containing raw materials include molasses and saccharified lignocellulosic biomass. Here, as lignocellulosic biomass, grassy and woody biomasses such as bagasse, corn stover, wheat straw, rice straw, switchgrass, napier grass, Erianthus, bamboo grass, and silvergrass, as well as waste wood, sawdust, bark, and waste paper can be suitably used. Lignocellulosic biomass contains cellulose and hemicellulose (hereinafter referred to as celluloses), and can be used as a raw material containing sugar by breaking down cellulose into sugar such as glucose and xylose using saccharification enzymes according to conventional methods.

The concentration of sugar in the medium may be 50 g/L to 500 g/L, preferably 60 g/L to 480 g/L, more preferably 60 g/L to 420 g/L, even more preferably 120 g/L to 420 g/L, particularly preferably 360 g/L to 420 g/L. When the culture medium contains sugar-containing materials, the sugar concentration converted from the concentration of the sugar-containing materials should be adjusted to be in the above range.

The medium preferably further contains a nitrogen source. The nitrogen source is not limited, but may be, for example, yeast extract, malt extract, meat extract, peptone, casamino acid, corn steep liquor, etc. Nitrogen sources may be ammonium sulfate, urea, potassium nitrate, etc.

Other than these, inorganic salts such as magnesium salts (magnesium sulfate heptahydrate, etc.), calcium salts (calcium chloride, etc.), phosphates (potassium phosphate, etc.), iron salts (iron sulfate, etc.), copper salts (copper sulfate, etc.) and sodium salts (sodium chloride, etc.) are also preferably contained. A higher C/N ratio (molar ratio of initial carbon source concentration to initial nitrogen source concentration in the medium, amount of carbon source/amount of nitrogen source) is desirable for high production of oils and fats, and is not particularly limited, for example, a C/N molar ratio of 100 or higher is preferred, 100 to 300 is more preferred, 200 to 300 is even preferred.

The culture temperature can be any temperature at which Metschnikowia sp. IS-391 or related strains can grow, and is not restricted, but for example, 10° C. to 30° C. is preferred and 20° C. to 28° C. is more preferred.

The pH of the medium can be any pH at which Metschnikowia sp. IS-391 or a related strain can grow, and is not restricted, for example, pH 4.5 or higher is preferred, pH 6.0 or lower is preferred, and about pH 5.5 is even preferred.

The culture method of Metschnikowia sp. IS-391 strain or its related strains is not restricted, and the strain may be cultured by directly inoculating the cells into the culture medium, or the pre-culture liquid obtained by pre-culturing may be inoculated into the liquid medium and cultured. Alternatively, the cells cultured on solid medium may be inoculated into the liquid medium and cultured. Known culture media used for conventional yeast culture may be used, such as PDA medium or YPD medium.

The method of culture is not restricted as long as Metschnikowia sp. IS-391 strain or its related strains can grow, for example, agitation culture, shaking culture, and static culture. The culture methods include, for example, batch culture, fed-batch culture, and continuous culture.

The culture time of Metschnikowia sp. IS-391 strain or its related strains can be determined according to the amount of fats and oils desired, for example, one day or longer, but four days or longer is preferred. For longer culture, it is preferable to supplement sugar midway through the culture. The upper limit of the culture period is not limited, but can be up to 30 days, for example.

The fats and oils can be collected from the cells after culture by conventional known methods. For example, the cells may first be separated from the culture medium by centrifugation, filtration, or other means, and then the fats and oils may be extracted from the cells with an organic solvent such as normal hexane.

The obtained fats and oils may be subjected to a refining process. The refining process may be a collective refining of fats and oils or a refining of a single component of fats and oils. For example, industrial methods of refining fats and oils can be applied, such as removing precipitated gums by adding water or acid to the fraction containing fats and oils obtained in the above collection process, removing free fatty acids by adding alkali, and decolorizing by using activated alumina. Silica gel chromatography may be repeated to further separate and refine the components in detail.

The resulting fats and oils may be used as-is or refined for use as edible fats and oils, or as raw materials for biofuels, bionaphtha, lubricating oil, or surfactants. Biodiesel can be obtained by performing the method of the present invention using biomass such as edible sugar, molasses, or saccharified lignocellulosic biomass as a raw material, and performing transesterification of the resulting fats and oils with methanol (FAME as product), or biodiesel, bio-jet fuel, and bionaphtha can be obtained by hydrogenation deoxygenation of the obtained fats and oils, followed by isomerization and, if necessary, cracking (HVO and HEFA as products). The obtained fats and oils can be co-processed with petroleum raw materials in a petroleum refinery's hydrotreating unit to obtain diesel, jet fuel, kerosene, gasoline, etc., as a mixture with petroleum fuels.

Lubricating oil can be used, for example, as a lubricating oil base material or additive, and can be obtained by performing the method of the present invention and using the resulting fats and oils as it is, partially hydrogenating the unsaturated bond, or generating fatty acids to form alkanes.

Surfactants include, for example, anionic surfactants such as fatty acid metal salts, which can be obtained by performing the method of the present invention, producing fatty acids from the resulting fats and oils, and neutralizing them.

### EXAMPLES

The invention will be specifically described below with examples, but the invention is not limited to the following embodiments.

### Example 1

<1> Acquisition of Metschnikowia sp. IS-391 Strain

Metschnikowia sp. IS-391 strain was obtained in Sodegaura City, Chiba Prefecture, Japan, from the flowers of Trifolium pretense (purple clover) using fats and oils production as an index.

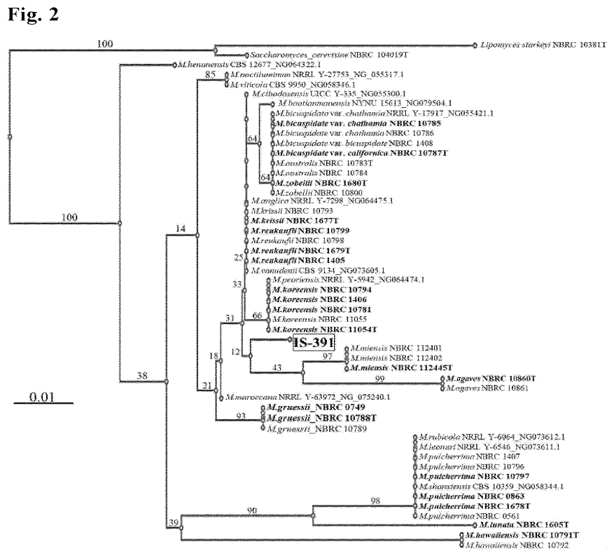

The DNA sequence of the D1/D2 region of the 26S rDNA region of the rRNA coding DNA of IS-391 strain was analyzed by DNA sequencing and phylogenetic analysis was performed with the sequence data of other strains of the genus Metschnikowia in the existing database. IS-391 did not form a monophyletic clade with any known species (FIG. 2). Therefore, IS-391 strain is presumed to be a new species in the genus Metschnikowia. The nucleotide sequence of the 26S rDNA D1/D2 region of the new IS-391 strain is shown below.

> IS-391_Metschnikowia sp.
(SEQ ID NO: 1)
ACGGCGAGTGAAGCGGCAAAAGCTCAAATTTGAAATCCTCCGGGAATTG
TAATTTGAAGGTTGTTGGTTGGAAAGATTACTTTAAGTCCATTGGAAAT
GGCGCCATGGAGGTGATAGCCCCCCGTAAAAGTAAACCCCCTCCCTTAA
TCCAAAACTTCCAAAGAGTCGAGTTGTTTGGGAATGCAGCTCTCTAAGT
GGGTGGTAAATTCCATCTAAAGCTAAATATATTGGCGAGAGACCGATAG
CGAACAAGTACAGTGATGGAAAGATGAAAAGCACTTTGAAAAGAAAGTG
AAAAGTACGTGAAATTGTTGAAAGGGAAGGCTTGCAAGCAGACACAACC
TCGGTTGGGCCAGCATCGGGAGTGGGGGAGATAAAAATGGGTAGGAATT
GTAGCTTTCTCTTAGAGTGTTATATCCTTCCTCTCTCATCCACCCCCTT
CCAGGCCTGCGATTCTCATCAGAGGATGCTGGCGTAATGGGTTGCAAGT
CGC

### Example 2

<2> Comparison with Other Metschnikowia Species

Each Metschnikowia sp. yeast strain grown on PDA (potato dextrose agar) was inoculated into YPD medium dispensed into 14 ml PS tubes and cultured for 48 hours at 24° C., 200 rpm with shaking (pre-culture). The pre-culture liquid was inoculated into 100 ml of sugarcane bagasse-saccharified medium in a 500 ml baffled flask to a final concentration of OD₆₀₀=0.1 and cultured with shaking at 28° C. and 160 rpm (main culture). Antifoam (Antifoam 204, Sigma) was added as needed. Sugarcane bagasse-saccharified liquid was prepared by crushing sugarcane bagasse into a few mm cubes, steam blasting (210° C., 5 min), and saccharification using cellulase.

The composition of each medium used for pre-culture is as follows:

### PDA Medium

24 g Potato Dextrose broth (Difco)
Agar (Wako) 20 g
Distilled water 1 L

### YPD Medium

20 g Bacto peptone (Difco)
10 g Yeast extract (Difco)
Glucose (Wako) 20 g

The composition of the medium used for triacylglyceride production culture is as follows:

### Sugarcane Bagasse-Saccharified Liquid Medium

Bacto peptone (Difco) 10 g
10 g Yeast extract (Difco)
Sugarcane bagasse-saccharified liquid added to reach a total concentration of 60 g/L of glucose and xylose
Meth up to 1 L distilled water

Samples were centrifuged (700 g, 5 min), and the precipitates were dried overnight in a freeze-dryer to determine the dry cell weight, which was then used for the triacylglyceride production measurement.

Triacylglyceride content was measured using the following method. To inactivate lipase in the dried cells, 260 μL of distilled water and 40 μL of 10× D-PBS (Wako) were added to resuspend the cells, which were then boiled for 10 minutes. Afterwards, 100 μL of 25 mg/mL Zymolyase solution (Nacalai Tesque, Zymolyase-20T) was added, and the mixture was incubated with shaking at 35° C. for 1 hour. Then, 200 μL of glass beads (Merck, G8772-500G) and 500 μL of hexane were added, and the mixture was shaken (TAITEC, E-36, at maximum speed) for 1 hour. After centrifugation (10,000 g, 10 minutes), the hexane layer was transferred to a new microtube, and an additional 500 μL of hexane was added and shaken for another hour. The hexane layer obtained after a second centrifugation (10,000 g, 10 minutes) was added to the microtube. The triacylglyceride content in the hexane extract was measured using an enzymatic assay (Lab Assay Triglyceride, Wako, 290-63701).

FIG. 3 shows the triacylglyceride production of each strain at 60 hours of main culture. It was found that the triacylglyceride production ability of IS-391 strain was significantly higher than that of other yeasts in the genus Metschnikowia.

### Example 3

### Evaluation of Added Nitrogen Sources Based on CYM Medium for IS-391 Strain

### [Test Tube Culture (Pre-Culture)]

Polystyrene (PS) tubes (15 mL, q17 mm×100 mm) from AS ONE containing 3 mL of modified YPD113 medium (10 g/L Bacto Yeast Extract, 10 g/L Bacto Tryptone, and 30 g/L glucose) were inoculated with the IS-391 oleaginous yeast strain. The yeast cells were gently scraped from a YPD agar plate (10 g/L Bacto Yeast Extract, 20 g/L Bacto Tryptone, and 20 g/L glucose) using the tip of a disposable loop. The rotary shaker NR-2 from TAITEC Corporation set at approximately 200 rpm was placed inside the SANYO INCUBATOR MIR-253, and shake culture was performed at 28° C. for one day.

[Evaluation of Nitrogen Sources Based on CYM Medium in a Triangular Flask Culture with 500 mL Baffle (Main Culture)]

CYM medium and modified CYM media, in which ammonium sulfate in the composition was replaced with either urea or potassium nitrate, were prepared (Table 1). The amount of ammonium sulfate, urea, or potassium nitrate added was adjusted to provide the same nitrogen concentration across all media. During preparation, each medium was adjusted to pH 6.0 using 6N KOH, then brought to the required volume, and sterilized by filtration through a 0.22 μm pore size MF filter. Subsequently, 100 mL of each medium was aseptically dispensed into sterilized 500 mL baffled Erlenmeyer flasks. The turbidity of the preculture in test tubes was measured using a spectrophotometer, and an amount was inoculated to achieve an OD600 nm value of 0.05 at the start of the main culture. Shake culture was then performed at 28° C. and 180 rpm.

TABLE 1
CYM-based medium for evaluation of added nitrogen sources

| | CYM medium | | | | CYM medium | |
|---|---|---|---|---|---|---|
| | Ammonium | | CYM medium | | Potassium | |
| | sulfate condition | | Urea condition | | nitrate condition | |
| Bacto Yeast extract | 0.75 | g/L | 0.75 | g/L | 0.75 | g/L |
| (NH₄)₂SO₄ | 0.1 | g/L | — | | — | |
| CO(NH₂)₂ | — | | 0.045 | g/L | — | |
| KNO₃ | — | | — | | — | |
| KH₂PO₄ | 0.4 | g/L | 0.4 | g/L | 0.15 | g/L |
| MgSO₄•7H₂O | 1.5 | g/L | 1.5 | g/L | 1.5 | g/L |
| CaCl₂•2H₂O | 0.22 | g/L | 0.22 | g/L | 0.22 | g/L |
| ZnSO₄ | 0.0003 | mg/L | 0.0003 | mg/L | 0.0003 | mg/L |
| MnSO₄•5H₂O | 0.029 | mg/L | 0.029 | mg/L | 0.029 | mg/L |
| CuSO₄•5H₂O | 0.025 | mg/L | 0.025 | mg/L | 0.025 | mg/L |
| MES (pH buffer) | 21.3 | g/L | 21.3 | g/L | 21.3 | g/L |
| Glucose | 60 | g/L | 60 | g/L | 60 | g/L |

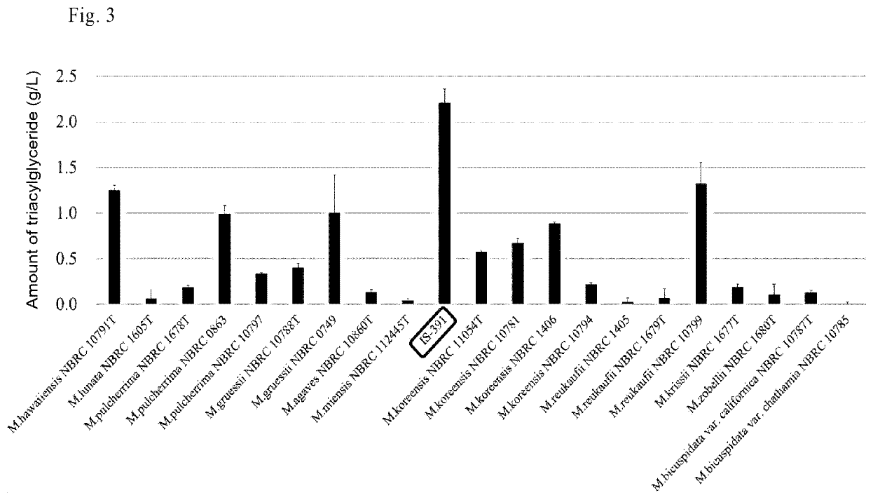

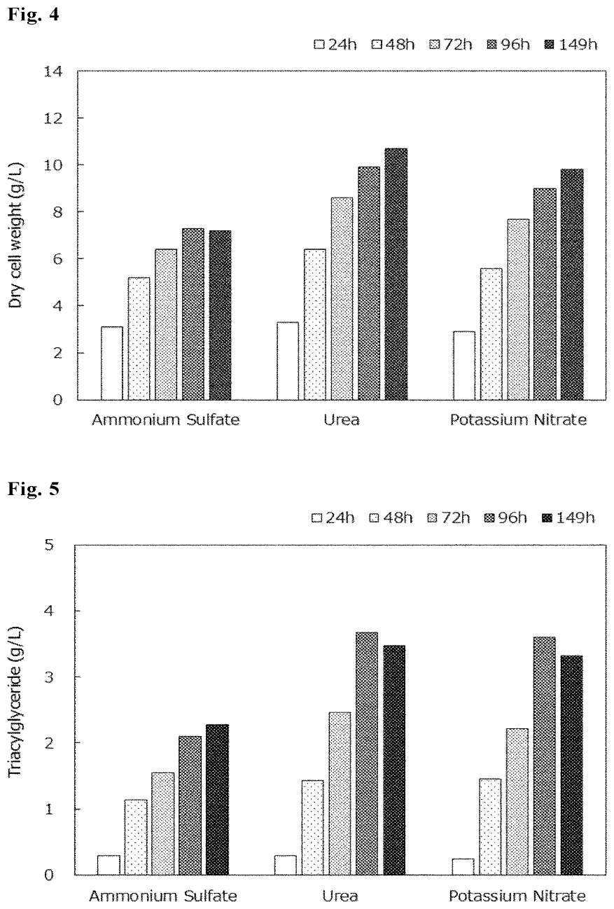

FIGS. 4 and 5 show the changes in dry cell weight and triacylglyceride production of IS-391 strain over time, respectively. Cell growth and triacylglyceride production were confirmed under each condition with ammonium sulfate, urea, and potassium nitrate as added nitrogen sources, respectively.

### Example 4

Evaluation of ammonium sulfate concentration in the culture medium in IS-391 strain

### [Evaluation of Ammonium Sulfate Concentration Based on CYM Medium in a 500 mL Baffled Triangular Flask Culture (Main Culture)]

Pre-culture was conducted under the same conditions as in Example 3. In main culture, CYM media were prepared with varying concentrations of ammonium sulfate: a standard concentration (ammonium sulfate concentration of 0.1 g/L, nitrogen concentration of 0.103 g/L, C/N ratio=273), 10 times the standard concentration (ammonium sulfate concentration of 1.0 g/L, nitrogen concentration of 0.294 g/L, C/N ratio=96), and 100 times the standard concentration (ammonium sulfate concentration of 10 g/L, nitrogen concentration of 2.202 g/L, C/N ratio=13) (Table 2). The C/N ratio refers to the molar ratio of carbon to nitrogen. Each medium was prepared in the same manner as described in Example 3, and shaking culture was carried out using baffled Erlenmeyer flasks under the same cultivation conditions.

TABLE 2
CYM-based medium for evaluating
ammonia sulfate concentration

| | CYM medium | | CYM medium | | CYM medium | |
|---|---|---|---|---|---|---|
| | 0.1 g/L | | 1.0 g/L | | 10 g/L | |
| | ammonium | | ammonium | | ammonium | |
| | sulfate | | sulfate | | sulfate | |
| Bacto Yeast extract | 0.75 | g/L | 0.75 | g/L | 0.75 | g/L |
| (NH₄)₂SO₄ | 0.1 | g/L | 1.0 | g/L | 10 | g/L |
| KH₂PO₄ | 0.4 | g/L | 0.4 | g/L | 0.4 | g/L |
| MgSO₄•7H₂O | 1.5 | g/L | 1.5 | g/L | 1.5 | g/L |
| CaCl₂•2H₂O | 0.22 | g/L | 0.22 | g/L | 0.22 | g/L |
| ZnSO₄ | 0.0003 | mg/L | 0.0003 | mg/L | 0.0003 | mg/L |
| MnSO₄•5H₂O | 0.029 | mg/L | 0.029 | mg/L | 0.029 | mg/L |
| CuSO₄•5H₂O | 0.025 | mg/L | 0.025 | mg/L | 0.025 | mg/L |
| MES (pH buffer) | 63.9 | g/L | 63.9 | g/L | 63.9 | g/L |
| Glucose | 60 | g/L | 60 | g/L | 60 | g/L |

*Total Nitrogen 10.9% BD Bionutrients-technical-manual

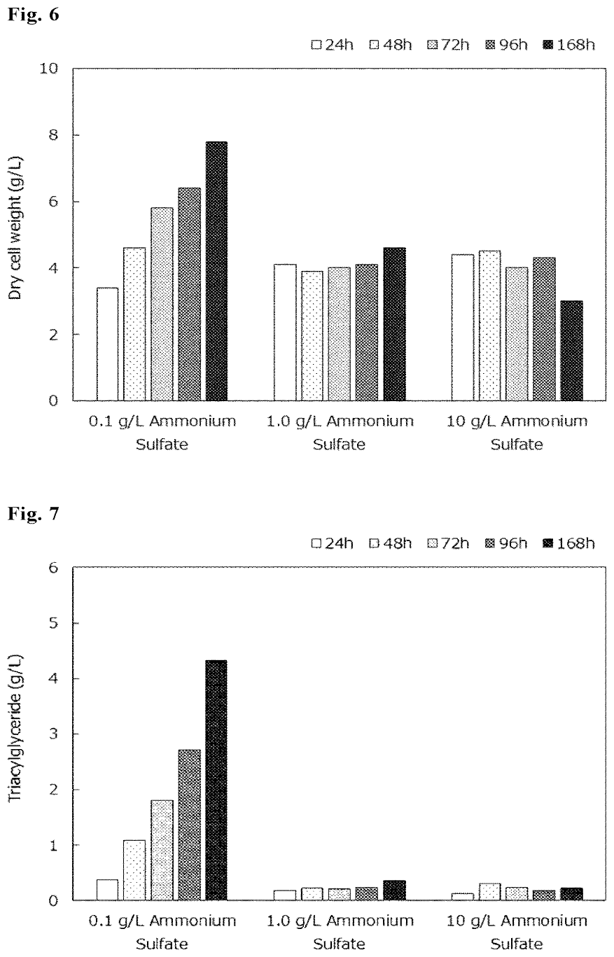

FIGS. 6 and 7 show the changes over time in the dry cell weight and triacylglyceride production of IS-391 strain under the medium conditions of each ammonium sulfate concentration, respectively. The highest triacylglyceride production of more than 4 g/L was observed at the standard ammonium sulfate concentration (0.1 g/L ammonium sulfate concentration, 0.103 g/L nitrogen concentration, C/N ratio=273), whereas triacylglyceride production was less than 1 g/L under the 10 times higher concentration (1.0 g/L ammonium sulfate concentration, 0.294 g/L nitrogen concentration, C/N ratio=96), and the 100 times higher concentration (10 g/L ammonium sulfate concentration, 2.202 g/L,

$$C / N ratio = 13 ) .$$

### Example 5

### Evaluation of Urea Concentration in the Culture Medium in IS-391 Strain

### [Evaluation of Urea Concentration Based on CYM Medium in a 500 mL Baffled Triangular Flask Culture (Main Culture)]

Pre-culture was conducted under the same conditions as in Example 3. In main culture, CYM media, in which ammonium sulfate was replaced with urea to achieve the same nitrogen concentration as described in Example 3, were prepared as follows: standard condition (urea concentration of 0.045 g/L, nitrogen concentration of 0.103 g/L, C/N ratio=273); 10 times the urea concentration (urea concentration of 0.45 g/L, nitrogen concentration of 0.294 g/L, C/N ratio=96); and 100 times the urea concentration (urea concentration of 4.5 g/L, nitrogen concentration of 2.202 g/L, C/N ratio=13) (Table 3). Apart from the medium composition, the preparation followed the same procedure as in Example 3, and 100 mL of each medium was prepared in sterilized 500 mL baffled Erlenmeyer flasks. Shaking cultivation was carried out under the same cultivation conditions as described in Example 3, using baffled Erlenmeyer flasks.

TABLE 3
CYM-based medium for evaluating urea concentration

| | CYM medium | | CYM medium | | CYM medium | |
|---|---|---|---|---|---|---|
| | 0.045 g/L Urea | | 0.45 g/L Urea | | 4.5 g/L Urea | |
| Bacto Yeast extract | 0.75 | g/L | 0.75 | g/L | 0.75 | g/L |
| CO(NH₂)₂ | 0.045 | g/L | 0. 45 | g/L | 4.5 | g/L |
| KH₂PO₄ | 0.4 | g/L | 0.4 | g/L | 0.4 | g/L |
| MgSO₄•7H₂O | 1.6 | g/L | 1.5 | g/L | 1.5 | g/L |
| CaCl₂•2H₂O | 0.22 | g/L | 0.22 | g/L | 0.22 | g/L |
| ZnSO₄ | 0.0003 | mg/L | 0.0003 | mg/L | 0.0003 | mg/L |
| MnSO₄•5H₂O | 0.029 | mg/L | 0.029 | mg/L | 0.029 | mg/L |
| CuSO₄•5H₂O | 0.025 | mg/L | 0.025 | mg/L | 0.025 | mg/L |
| MES (pH buffer ) | 63.9 | g/L | 63.9 | g/L | 63.9 | g/L |
| Glucose | 60 | g/L | 60 | g/L | 60 | g/L |

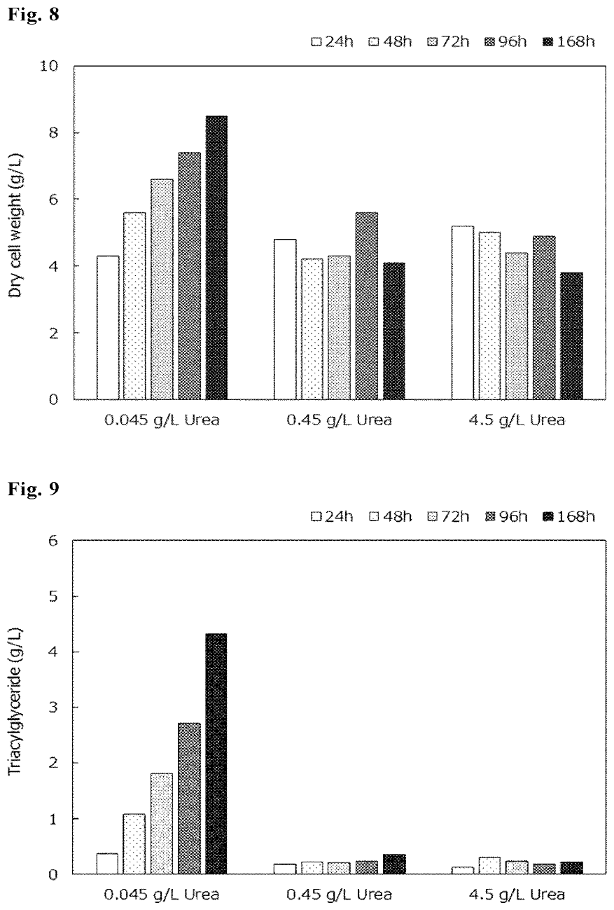

FIGS. 8 and 9 show the changes over time in the dry cell weight and triacylglyceride production of IS-391 strain under the medium conditions of each urea concentration, respectively. The highest triacylglyceride production of more than 4 g/L was observed under the standard condition (urea concentration of 0.045 g/L, nitrogen concentration of 0.103 g/L, C/N ratio=273), whereas triacylglyceride production was less than 1 g/L under the 10 times higher concentration (urea concentration of 0.45 g/L, nitrogen concentration of 0.294 g/L, C/N ratio=96), and the 100 times higher concentration (urea concentration of 4.5 g/L, nitrogen concentration of 2.202 g/L, C/N ratio=13).

### Example 6

### Evaluation of Incubation Temperature in Flask Culture

### [Test Tube Culture (Pre-Culture)]

YEL medium (3.5 mL; 5 g/L Bacto Yeast Extract, and 30 g/L glucose) was dispensed into 15 mL polystyrene (PS) tubes (φ17 mm×100 mm) from AS ONE. The IS-391 strain was scraped from frozen cell stock using a loop and inoculated into the medium. Shaking culture was conducted at 28° C. with the TAITEC Rotary Shaker NR-2 set to approximately 200 rpm, placed inside the SANYO INCUBATOR MIR-253, for 1 day. Under the same conditions, culture was performed in three separate test tubes.

[Evaluation of Incubation Temperature in a Triangular Flask Culture with 500 mL Baffle (Main Culture)]

For main culture, as in Example 3, CYM medium (0.1 g/L ammonium sulfate, pH 6.5) was prepared, and 97 mL of the medium was dispensed into sterilized 500 mL baffled Erlenmeyer flasks. The culture temperature conditions were set to 24° C., 28° C., and 32° C. The preculture liquid from test tube culture was mixed, and 3 mL of the preculture liquid was inoculated into each flask. Shaking culture was then carried out at 180 rpm.

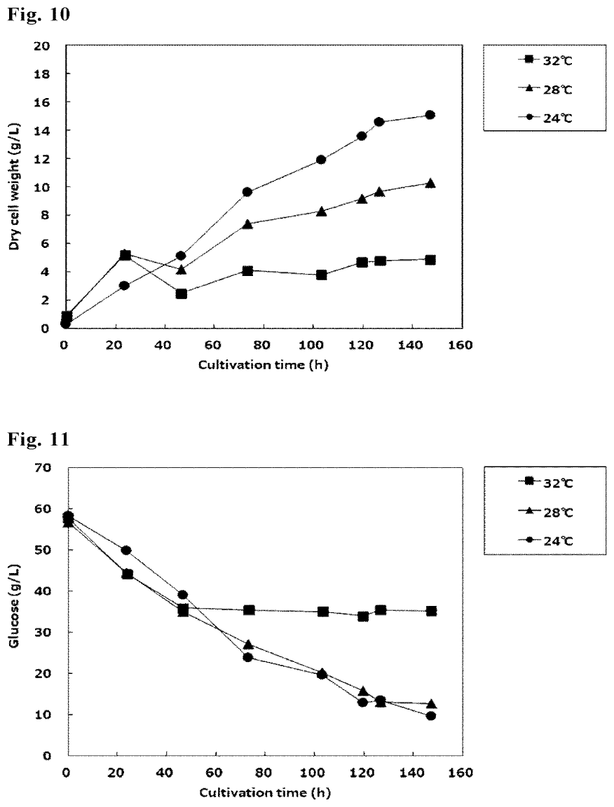

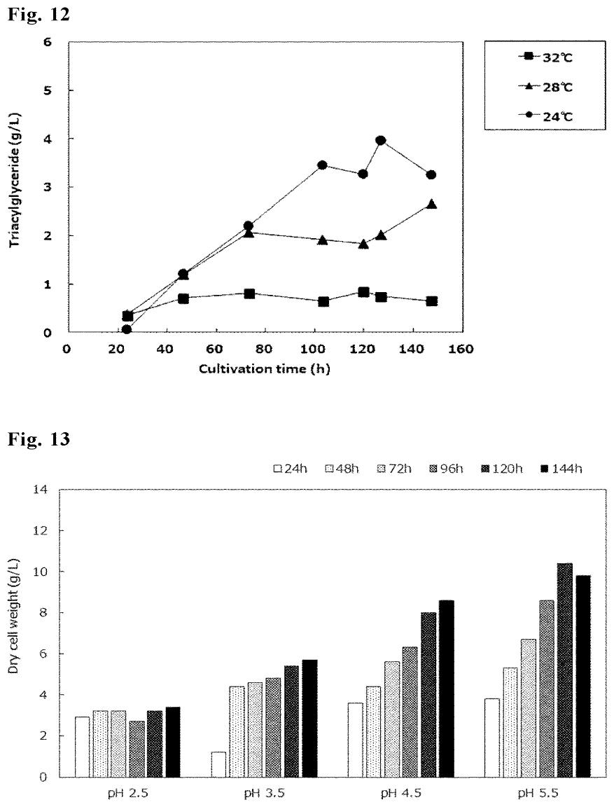

FIGS. 10, 11, and 12 show the changes over time in the dry cell weight, glucose concentration in the medium, and triacylglyceride production of IS-391 strain at various temperature conditions, respectively. Cell growth, glucose consumption, and triacylglyceride production were observed at incubation temperatures of 24° C., 28° C., and 32° C. At 24° C., the triacylglyceride production rate and triacylglyceride production were better. At 32° C., both growth and triacylglyceride production were significantly lower.

### Example 7

### Evaluation of Culture pH in Jar Fermenter Culture

### [Test Tube Culture (Pre-Culture)]

YPD medium (3.5 mL per tube) was dispensed into 15 mL polystyrene (PS) tubes (φ17 mm×100 mm) from AS ONE. The IS-391 strain, grown on a YPD agar plate, was gently scraped with the tip of a disposable loop and inoculated into the medium. The tubes were placed in a SANYO INCUBATOR MIR-253, equipped with a TAITEC ROTARY SHAKER NR-2 set at approximately 200 rpm, and shaking culture was carried out at 28° C. for 1 day.

### [Evaluation of pH in 250 mL Jar Fermenter Culture (Main Culture)]

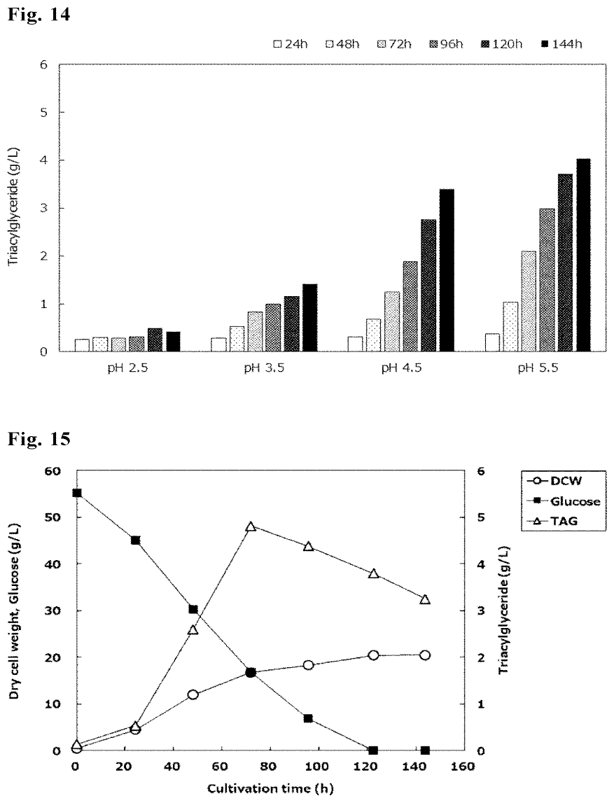

The jar fermenter was an ABLE-Biott Bio Jr. 8 250 mL 8-cell culture tank, and compressed air supplied as a utility was used for air supply. The main culture tanks were filled with 100 mL of CYM medium with reagent glucose as the sugar source. The sugar concentration was set at approximately 60 g/L (after inoculation). The culture pH conditions were pH 2.5, 3.5, 4.5 and pH 5.5. After inoculating a quantity of pre-culture liquid with an OD600 nm value of 0.1 at the start of the main culture, batch culture at 28° C. was started. The lower limit of the stirring speed was set at 300 rpm, and the stirring speed was automatically controlled (DO cascade control) so that the lower limit of DO was 2 ppm. Aeration conditions were set at 100 mL/min (1 vvm). After the start of culture, 0.5 N KOH was used to automatically control the pH conditions for each study. FIGS. 13 and 14 show the changes over time in the dry cell weight and triacylglyceride production of IS-391 strain at each culture pH condition, respectively. A trend of decreasing triacylglyceride production was observed as the culture pH conditions decreased.

### Example 8

### Jar Fermenter Culture (24° C., pH 5.5)

### [250 mL Jar Fermenter Culture, 24° C., pH 5.5 Conditions (Main Culture)

Main culture experiments were conducted under the same conditions as in Example 7, with pH conditions at pH 5.5 and culture temperature at 24° C. FIG. 15 shows the changes over time in dry cell weight, glucose concentration, and triacylglyceride production of IS-391 strain. Triacylglyceride production reached a maximum of 4.8 g/L, and the yield of triacylglyceride to sugar, determined from the sugar consumption, was 12%.

### Example 9

### Fed-Batch Culture in Jar Fermenter

### [Test Tube Culture (Pre Pre-Culture)]

After inoculating into 3.5 mL of YEL medium contained in a 15 mL polystyrene (PS) tube (φ17 mm×100 mm, manufactured by AS ONE) the IS-391 strain obtained by scraping frozen cell stock using a loop, the tubes were placed in a SANYO INCUBATOR MIR-253 equipped with a TAITEC ROTARY SHAKER NR-2, set to approximately 200 rpm. Shaking culture was carried out at 28° C. for 1 day. The test tube culture was performed three times under the same conditions.

### [Flask Culture (Pre-Culture)]

After inoculating 3 mL of test tube culture liquid into 100 mL of YEL medium contained in a 500 mL baffled Erlenmeyer flask, the flasks were placed on an Iwashiya Bioscience four-station orbital shaker set to 180 rpm. The culture was carried out at 28° C. for 1 day. The flask culture was performed three times under the same conditions.

### [2 L Jar Fermenter Flow Culture (Main Culture)]

In main culture, CYM medium (0.1 g/L ammonium sulfate) was used as the initial medium, and feed medium prepared based on the CYM medium composition was used (Table 4).

TABLE 4
Initial medium and feed medium

| | Initial medium | | Feed medium | |
|---|---|---|---|---|
| Bacto Yeast extract | 0.75 | g/L | 15.6 | g/L |
| (NH₄)₂SO₄ | 0.1 | g/L | 1.0 | g/L |
| KH₂PO₄ | 0.4 | g/L | 8.4 | g/L |
| MgSO₄•7H₂O | 1.5 | g/L | 31.4 | g/L |
| CaCl₂•2H₂O | 0.22 | g/L | 4.6 | g/L |
| ZnSO₄ | 0.0003 | mg/L | 0.0063 | mg/L |
| MnSO₄•5H₂O | 0.029 | mg/L | 0.605 | mg/L |
| CuSO₄•5H₂O | 0.025 | mg/L | 0.523 | mg/L |
| Glucose | 20 | g/L | 625 | g/L |

The jar fermenter was a 2 L Bioneer-Neo 2 L culture tank manufactured by Marubishi Bioengineering, and compressed air supplied as a utility was used for air supply. After mixing the pre-culture liquid from each flask culture and inoculating 200 mL of it, the culture was started at 28° C. The agitation speed at the start was 500 rpm. At the start of incubation, the agitation speed was fixed at 500 rpm and the aeration condition was 1.0 L/min (1 vvm). After the start of culture, 1 N KOH was used to control the lower limit of pH 5.5. After confirming the decreasing trend of glucose concentration in the culture medium, the agitation speed was reduced to 360 rpm under manual control. After 28 hours of incubation, the oxygen consumption rate of the cells decreased due to the decrease in glucose concentration in the initial medium, and the DO value increased. This phenomenon was taken as the timing for the start of feeding and the supply of flow-through medium was started. The DO sensor detected a phenomenon in which the dissolved oxygen (DO) concentration increased when glucose was depleted, and the culture was shifted to the DO stat control (DO 22% upper limit control), in which automatic continuous feeding was performed. Considering cell growth, the agitation speed was increased manually in a stepwise manner at various intervals during the fed-batch culture. Considering the remaining glucose concentration, feeding was suspended after 51 and 145 hours of culture, and resumed after 52 and 148 hours of culture, respectively. Fed-batch culture was continued until 192 hours of culture.

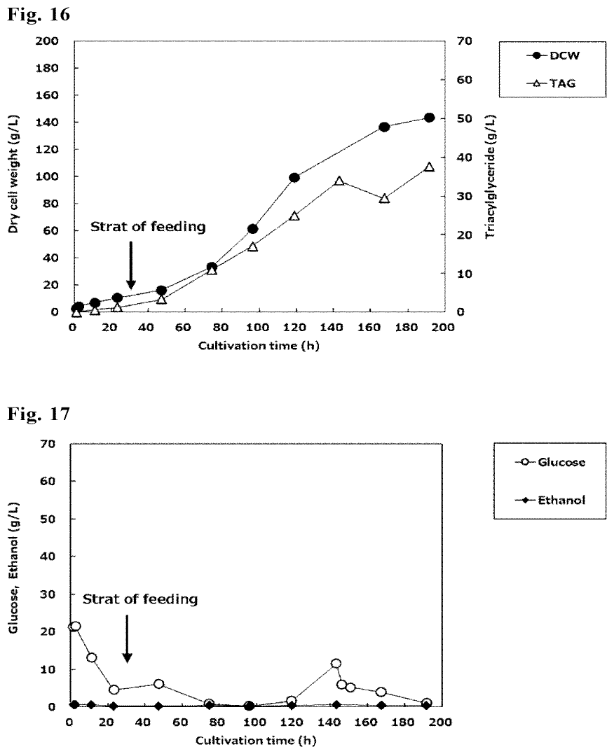

FIGS. 16 and 17 show the changes over time in dry cell weight, triacylglyceride production, glucose concentration, and ethanol concentration of IS-391 strain in fed-batch culture, respectively. After the start of culture, the concentration of cells continued to increase until the end of culture after 192 hours, reaching a maximum dry cell weight of 143.5 g-dcw/L. The amount of triacylglyceride produced per culture medium also increased with the cell growth, reaching a maximum of 37.5 g/L after 192 hours of culture.

### Example 10

### Evaluation of the Initial Sugar Concentration in the Medium in Jar Fermenter Culture

### [Test Tube Culture (Pre-Culture)]

IS-391 strain, grown on a YPD agar plate, was lightly scraped using the tip of a disposable inoculation loop and inoculated into 3.5 mL of YEL medium contained in a 15 mL polystyrene (PS) tube (φ17 mm×100 mm, manufactured by AS ONE). The tubes were placed on an Iwashiya Bioscience Multi-Station Orbital Shaker (MLU-4-GR-16), set to approximately 140 rpm, and incubated at 24° C. with shaking for 1 day. A total of 15 test tube cultures were performed under the same conditions.

### [Culture in 250 mL Jar Fermenter Culture (Main Culture)]

The same jar fermenters and air supply were used as in Example 7. A modified CYM medium (CYM16 medium; Table 5) was utilized as the culture medium. The initial glucose concentration in the media was set to three levels: 60 g/L (standard), 120 g/L, and 360 g/L. For the medium composition under the 60 g/L glucose condition (Table 5), it served as the standard. For media with higher glucose concentrations, the concentrations of other medium components were proportionally increased based on the excess glucose concentration relative to the standard (60 g/L condition). A total of 118.8 mL of each medium was poured into a 250 mL culture vessel. Pre-culture liquid (6.3 mL), prepared by mixing test tube culture liquids, was inoculated into each fermenter vessel, and batch culture was initiated at 24° C. under different glucose concentration conditions. Automatic agitation speed control was employed using DO cascade control to maintain a DO lower limit of 2 ppm. The aeration rate was set to 125 mL/min (1 vvm). pH was automatically controlled to 5.5 using 1.0 N KOH and 0.5 N H₂SO₄ throughout the culture process.

TABLE 5
CYM16 medium

| | CYM16 medium | |
|---|---|---|
| Corn steep liquor | 8.0 | g/L |
| (Sigma-Aldrich) | | |
| (NH₄)₂SO₄ | 0.2 | g/L |
| KH₂PO₄ | 0.4 | g/L |
| MgSO₄•7H₂O | 1.5 | g/L |
| CaCl₂•2H₂O | 0.22 | g/L |
| ZnSO₄ | 0.0003 | mg/L |
| MnSO₄•5H₂O | 0.029 | mg/L |
| CuSO₄•5H₂O | 0.025 | mg/L |
| Glucose | 60 | g/L |

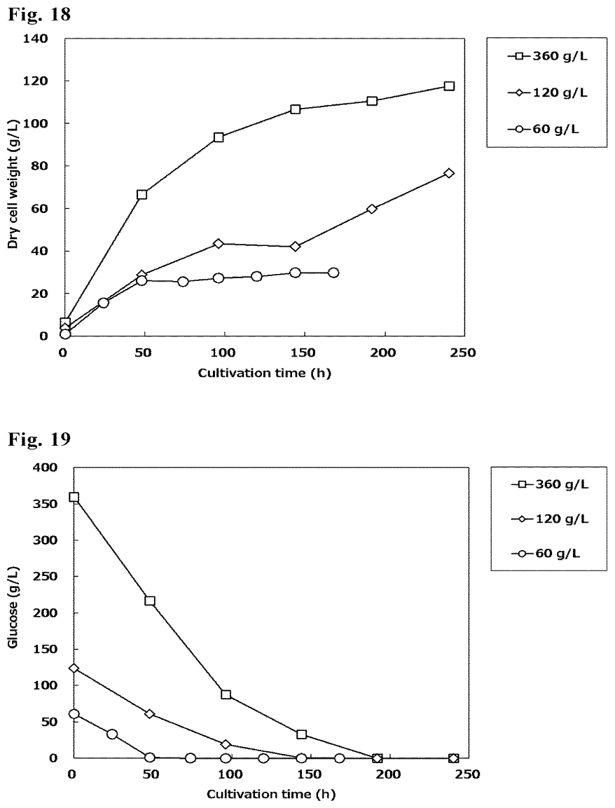

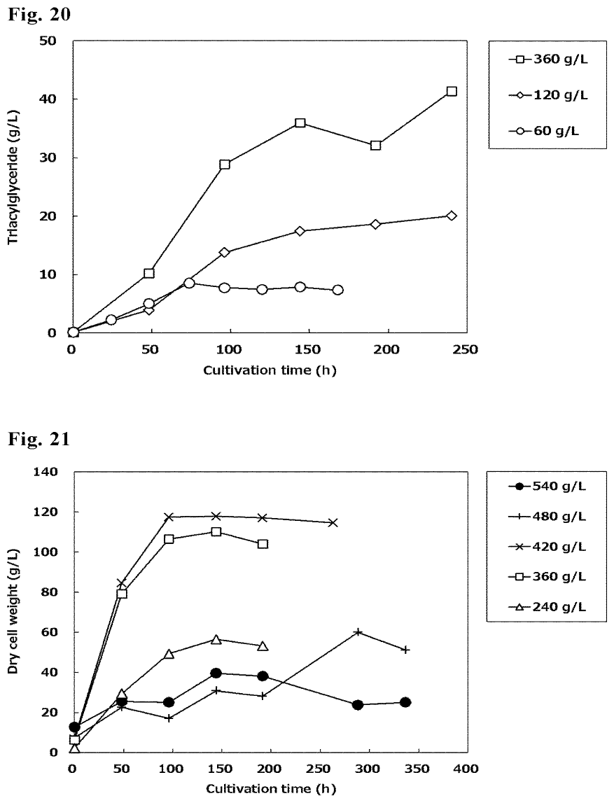

FIGS. 18, 19, and 20 show the changes over time in dry cell weight, glucose concentration, and triacylglyceride production of IS-391 strain under each glucose concentration condition, respectively. Cell growth and triacylglyceride production were observed in the glucose concentration range of 60 g/L to 360 g/L.

### Example 11

### Evaluation of Initial Sugar Concentration in the Medium in Jar Fermenter Culture2

### [Test Tube Culture (Pre-Culture)]

IS-391 strain, grown on a YPD agar plate, was lightly scraped using the tip of a disposable inoculation loop and inoculated into 3.5 mL of YEL medium contained in a 15 mL polystyrene (PS) tube (φ17 mm×100 mm, manufactured by AS ONE). The tubes were placed in a SANYO INCUBATOR MIR-151 equipped with a TAITEC ROTARY SHAKER NR-2, set to approximately 200 rpm, and incubated at 24° C. with shaking for 1 day. A total of 20 test tube cultures were performed under the same conditions.

### [Culture in 250 mL Jar Fermenter Culture (Main Culture)].

The same jar fermenters and air supply were used as in Example 7. The initial glucose concentrations in the media were set to 240 g/L, 360 g/L, 420 g/L, 480 g/L, and 540 g/L, respectively. The medium composition for the 60 g/L glucose condition from Example 10 (Table 5) was used as the standard. For each glucose concentration condition, the concentrations of other medium components were proportionally increased based on the additional glucose concentration relative to the standard (60 g/L condition).

For culture, 152 mL of each medium was poured into a 250 mL culture vessel. Pre-culture liquid (8 mL), prepared by mixing test tube culture liquids, was inoculated into each vessel. Batch cultivation was initiated at 24° C. under different glucose concentration conditions. Automatic agitation speed control was employed using DO cascade control to maintain a DO lower limit of 2 ppm. The aeration rate was set to 150 mL/min. Throughout the cultivation process, pH was automatically controlled to remain at 5.5 using 1.0 N KOH and 0.5 N H₂SO₄.

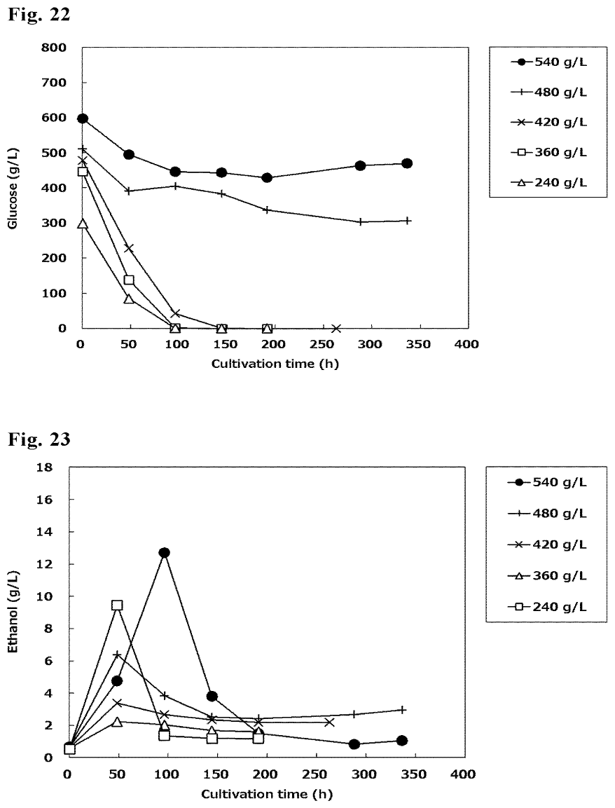

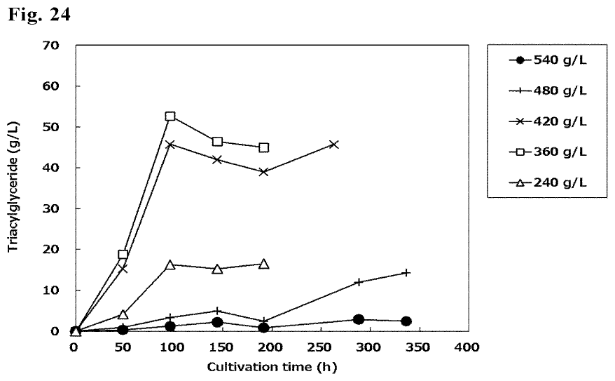

FIGS. 21, 22, 23, and 24 show the changes over time in dry cell weight, glucose concentration, ethanol concentration, and triacylglyceride production of IS-391 strain under each glucose concentration condition, respectively. Under the conditions set for each glucose concentration, the actual measured glucose concentrations were observed to be 7 to 25% higher than the intended values. For the initial set glucose concentrations of 240 g/L, 360 g/L, 420 g/L, 480 g/L, and 540 g/L, the measured values were 300 g/L, 447 g/L, 479 g/L, 512 g/L, and 598 g/L, respectively. Cell growth and triacylglyceride production were observed over the range of actual measured glucose concentrations from 300 g/L to 512 g/L.

1. A method of producing a fat and an oil, comprising:
culturing Metschnikowia sp. IS-391 strain (NITE BP-03676) or a related strain thereof in a medium containing sugar to produce a fat and an oil from the sugar; and
collecting the resulting fat and oil.

2. The method according to claim 1, wherein the medium containing sugar is a medium containing saccharified product of lignocellulosic biomass.

3. The method according to claim 1, wherein the culture temperature is between 10° C. and 30° C.

4. The method according to claim 1, wherein the molar ratio (C/N ratio) of the concentration of initial carbon source to the concentration of initial nitrogen source in the medium is greater than 100.

5. The method according to claim 1, wherein the related strain is a strain whose sequence of the 26S rDNA D1/D2 region is 98% or more identical to SEQ ID NO: 1 and has fat and oil production ability equivalent to that of IS-391 strain.

6. The method according to claim 1, wherein the fat and oil are raw materials for biofuels, bionaphtha, food oil, lubricating oil, or surfactants.

7. Oils and fats produced by the method of claim 1.

8. A biofuel or bionaphtha made from the fats and oils of claim 7.

9. A Metschnikowia sp. IS-391 strain (NITE BP-03676).


SEQUENCE LISTING (1 sequence)

SEQ ID NO: 1 - DNA, 490 bp, Metschnikowia sp.
  source 1..490: mol_type = genomic DNA
acggcgagtgaagcggcaaaagctcaaatttgaaatcctccgggaattgtaatttgaaggttgttttggttgggaaagattactttaagtccattggaaaatggcgccatggagggtgatagccccgtaaaagtaaaccccctcccttaatccaaaacttccaaagagtcgagttgtttgggaatgcagctctaagtgggtggtaaattccatctaaagctaaatattggcgagagaccgatagcgaacaagtacagtgatggaaagatgaaaagcactttgaaaagaaagtgaaaaagtacgtgaaattgttgaaagggaagggcttgcaagcagacacaacctcggttgggccagcatcggagtggggggagataaaaatgggtaggaatgtagctttctttagagtgttatatccttcccttccatctccacccccttccgaggcctgcgattcttcaaggatgctggcgtaatggttgcaagtcgc